(19) United States
(12) Patent Application Publication
(10) Pub. No.: US 2023/0226326 A1
(43) Pub. Date: Jul. 20, 2023

(54) ELONGATED CATHETER ASSEMBLY HAVING GUIDEWIRE DEFLECTOR

(71) Applicant: Boston Scientific Medical Device Limited, Galway (IE)

(72) Inventors: Linus Leung, Toronto (CA); Robert Galvin, Toronto (CA)

(21) Appl. No.: 18/187,371

(22) Filed: Mar. 21, 2023

Related U.S. Application Data
(63) Continuation of application No. PCT/IB2021/057790, filed on Aug. 25, 2021.
(60) Provisional application No. 63/083,195, filed on Sep. 25, 2020.

(51) Int. Cl. A61M 25/09

(52) U.S. Cl. CPC A61M 25/09041; A61M 2025/09008; A61M 2205/0288

(57) ABSTRACT

An elongated catheter assembly defines a catheter lumen configured to receive the distal guidewire section of the guidewire, and also defines an axial portal and a radial portal. A guidewire deflector is mounted to the elongated catheter assembly. The guidewire deflector is configured to selectively deflect urged axial movement of the distal guidewire section of the guidewire away from the axial portal and radially toward the radial portal.

### CROSS REFERENCE TO RELATED APPLICATIONS:

This application claims the benefit of International Application Number PCT/IB2021/057790, entitled “ELONGATED CATHETER ASSEMBLY HAVING GUIDEWIRE DEFLECTOR,” and filed Aug. 25, 2021, which claims the benefit of U.S. Provisional Application No. 63/083,195, entitled “ELONGATED CATHETER ASSEMBLY HAVING GUIDEWIRE DEFLECTOR,” and filed Sep. 25, 2020, which are hereby incorporated by reference in their entireties.

### TECHNICAL FIELD

This document relates to the technical field of (and is not limited to) an elongated catheter assembly for use with a guidewire, and having a guidewire deflector (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with existing (known) medical catheter assemblies. After much study of, and experimentation with, the existing (known) medical catheter assemblies, an understanding (at least in part) of the problem and its solution have been identified (at least in part) and are articulated (at least in part) as follows:

The known re-entry catheter may rely on the ability of the physician to orient a guidewire such that the distal tip of the guidewire is pointing out of the side port of the re-entry catheter. The process to orient the distal tip may be challenging due to the manipulations (whipping, turning, torqueing) of the guidewire. Also, determining whether the distal tip (of the guidewire) is towards the side port may also be difficult due to the imaging resolution (of a medical image generated by a medical imaging system).

It may be desirable to provide a guidewire deflector configured to selectively deflect urged axial movement of the guidewire away from an axial portal (frontal port) and radially toward a radial portal (side port).

To mitigate, at least in part, at least one problem associated with the existing technology, there is provided (in accordance with a major aspect) an apparatus. The apparatus is for use with a guidewire having a distal guidewire section. The apparatus includes and is not limited to (comprises) an elongated catheter assembly defining a catheter lumen configured to receive the distal guidewire section of the guidewire. The catheter lumen also defines an axial portal and a radial portal. A guidewire deflector is mounted to the elongated catheter assembly. The guidewire deflector is configured to selectively deflect urged axial movement of the distal guidewire section of the guidewire away from an axial portal and radially toward the radial portal.

To mitigate, at least in part, at least one problem associated with the existing technology, there is provided (in accordance with a major aspect) an apparatus. The apparatus is for use with a guidewire having a distal guidewire section. The apparatus includes and is not limited to (comprises) an elongated catheter assembly defining a catheter lumen extending axially along the elongated catheter assembly. The catheter lumen is configured to receive the distal guidewire section of the guidewire. The elongated catheter assembly defines an axial portal extending axially from the catheter lumen. The elongated catheter assembly also defines a radial portal extending radially from the catheter lumen. A guidewire deflector is mounted in the catheter lumen. The guidewire deflector is configured to selectively deflect urged axial movement of the distal guidewire section of the guidewire (which is being made to travel axially along the catheter lumen) away from an axial portal and radially toward the radial portal.

To mitigate, at least in part, at least one problem associated with the existing technology, there is provided (in accordance with a major aspect) a method. The method is for using a guidewire having a distal guidewire section. The method includes and is not limited to (comprises) using a guidewire deflector mounted to an elongated catheter assembly (defining a catheter lumen configured to receive the distal guidewire section of the guidewire, and also defines an axial portal and a radial portal) to selectively deflect urged axial movement of the distal guidewire section of the guidewire away from an axial portal and radially toward the radial portal.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 to FIG. 4 depict a perspective side view (FIG. 1) and cross-sectional views (FIG. 2 to FIG. 4) of embodiments (implementations) of an elongated catheter assembly having a guidewire deflector; and

FIG. 5 and FIG. 6 depict cross-sectional views of embodiments (implementations) of the guidewire deflector of FIG. 1; and

FIG. 7 to FIG. 9 depict cross-sectional views of embodiments (implementations) of the guidewire deflector of FIG. 1; and

FIG. 10 to FIG. 12 depict cross-sectional views of embodiments (implementations) of the guidewire deflector of FIG. 1; and

FIG. 13 to FIG. 16 depict cross-sectional views (FIG. 13 to FIG. 15) of embodiments (implementations) of the guidewire deflector of FIG. 1, and a side view (FIG. 16) of embodiments (implementations) of the elongated catheter assembly of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

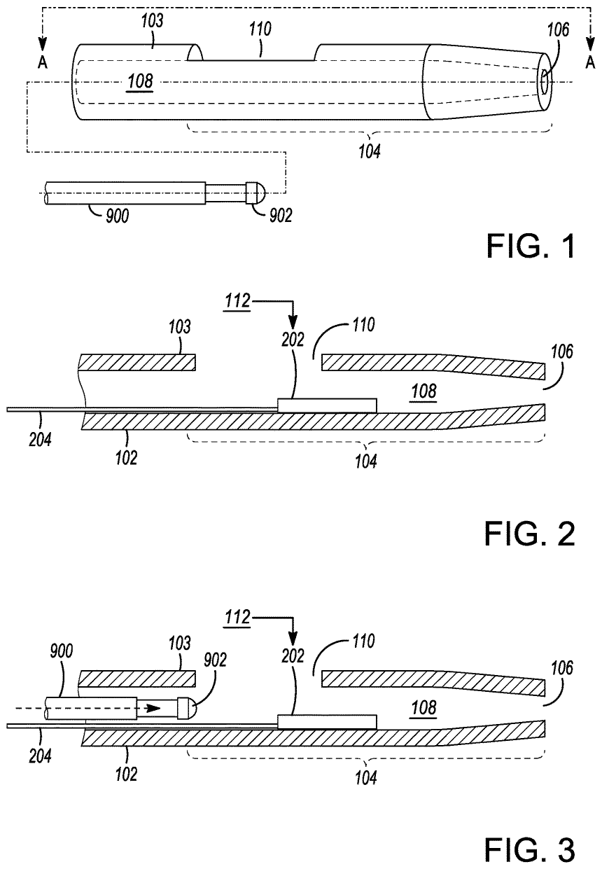

FIG. 1 to FIG. 6 depict a perspective side view (FIG. 1) and cross-sectional views (FIG. 2 to FIG. 6) of embodiments (implementations) of an elongated catheter assembly 102 having a guidewire deflector 112. The cross-sectional views (FIG. 2 to FIG. 6) are taken along a cross-sectional line A-A of FIG. 1.

Referring to the embodiment (implementation) as depicted in FIG. 1, the elongated catheter assembly 102 is configured to be used with a guidewire 900 having a distal guidewire section 902. The elongated catheter assembly 102 defines a catheter lumen 108. The catheter lumen 108 extends (axially) along an elongated length of the elongated catheter assembly 102. A proximal section (positioned on the left side of FIG. 1) of the catheter lumen 108 is configured to receive the distal guidewire section 902 of the guidewire 900. A distal section (which is positioned on the right side of FIG. 1) of the catheter lumen 108 defines an axial portal 106 and a radial portal 110 (also called a side port).

The catheter lumen 108, the axial portal 106 and the radial portal 110 are in fluid communication with each other (one another). The axial portal 106 is positioned axially relative to the catheter lumen 108. The radial portal 110 is positioned radially relative to the catheter lumen 108. The guidewire 900 is configured to be movable (that is, urged for axial movement) along a length of the catheter lumen 108 towards (and through) the axial portal 106 (that is, from the proximal section (which is located on the left side of FIG. 1) to the distal section of the guidewire 900 (which is located on the right side of FIG. 1). The elongated catheter assembly 102 is configured to be inserted into a confined space defined by a living body (the patient).

Referring to the embodiment (implementation) as depicted in FIG. 1, the catheter lumen 108 extends axially along (an elongated length of) the elongated catheter assembly 102. The axial portal 106 extends axially from (the distal section of) the catheter lumen 108. The radial portal 110 extends radially from (the distal section of) the catheter lumen 108. The axial portal 106 and the radial portal 110 are positioned, preferably, proximate to each other (one another).

Referring to the embodiments (implementations) as depicted in FIG. 2 to FIG. 6, the elongated catheter assembly 102 incudes the guidewire deflector 112. The guidewire deflector 112 is, preferably, user controllable (actuatable). For the case where the guidewire deflector 112 is not utilized (is not activated), the distal guidewire section 902 of the guidewire 900 may be urged to move toward and reach (extend from) the axial portal 106, thereby allowing the distal guidewire section 902 to by-pass the radial portal 110 and be urged to move through (and extend from) the axial portal 106. The guidewire deflector 112 is configured to selectively deflect movement (once activated to do so) of the distal guidewire section 902 of the guidewire 900 toward the radial portal 110; in this arrangement, therefore, urged movement of the distal guidewire section 902 toward (and extension from) the axial portal 106 may be averted (in response to activation of the guidewire deflector 112).

Referring to the embodiment (implementation) as depicted in FIG. 2 to FIG. 6, the components of the elongated catheter assembly 102 and/or the guidewire deflector 112 include biocompatible material properties suitable for sufficient performance (such as electric dielectric strength, thermal performance, electrical insulation, corrosion, water resistance, heat resistance) for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment (implementation) as depicted in FIG. 2 to FIG. 6, the guidewire deflector 112 includes a balloon assembly 202. The balloon assembly 202 is configured to be selectively inflated and deflated. An inflation lumen 204 is mounted to (supported by) the elongated catheter assembly 102. The inflation lumen 204 extends along a length of the elongated catheter assembly 102. The inflation lumen 204 is in fluid communication with the balloon assembly 202. The inflation lumen 204 extends from the proximal section of the elongated catheter assembly 102. The proximal section of the inflation lumen 204 is configured to be fluidly connected to an inflation source (known and not depicted). It will be appreciated that the inflation lumen 204 may be positioned in the side wall of the elongated catheter assembly 102. It will be appreciated that the inflation lumen 204 may be positioned in the catheter lumen 108 (if so desired). It will be appreciated that the elongated catheter assembly 102 may provide a multi-lumen shaft to facilitate selective inflation of the balloon assembly 202.

Referring to the embodiments (implementations) as depicted in FIG. 1 and FIG. 2, the elongated catheter assembly 102 has a sidewall 103. The elongated catheter assembly 102 also has a distal section 104 extending from the sidewall 103. The catheter lumen 108 extends axially toward, and through, the distal section 104 to the axial portal 106. The distal section 104 is configured to permit (facilitate) movement of the distal guidewire section 902 of the guidewire 900 travelling axially along the catheter lumen 108 toward, and through, the axial portal 106. The distal section 104, which defines the radial portal 110, extends radially from the catheter lumen 108. The radial portal 110 extends radially through the sidewall 103.

Referring to the embodiment (implementation) as depicted in FIG. 2, the guidewire deflector 112 is (generally) mounted to the elongated catheter assembly 102. The guidewire deflector 112 is configured to selectively deflect urged axial movement of the distal guidewire section 902 (of the guidewire 900) away from an axial portal 106 and radially toward the radial portal 110 (once activated accordingly).

Referring to the embodiment (implementation) as depicted in FIG. 2, the guidewire deflector 112 is (preferably) mounted in the catheter lumen 108 (at the distal section of the catheter lumen 108). The guidewire deflector 112 is configured to selectively deflect urged axial movement of the distal guidewire section 902 of the guidewire 900 away from the axial portal 106 and radially toward the radial portal 110 (normally, the guidewire 900 is made to travel axially along the catheter lumen 108 toward the axial portal 106).

Referring to the embodiment (implementation) as depicted in FIG. 2, the guidewire deflector 112 is mounted (positioned) in the catheter lumen 108 at the distal section 104.

The guidewire deflector 112 is also mounted (positioned) proximately to the axial portal 106.

Referring to the embodiment (implementation) as depicted in FIG. 3, the distal guidewire section 902 (of the guidewire 900) is urged to move toward the axial portal 106.

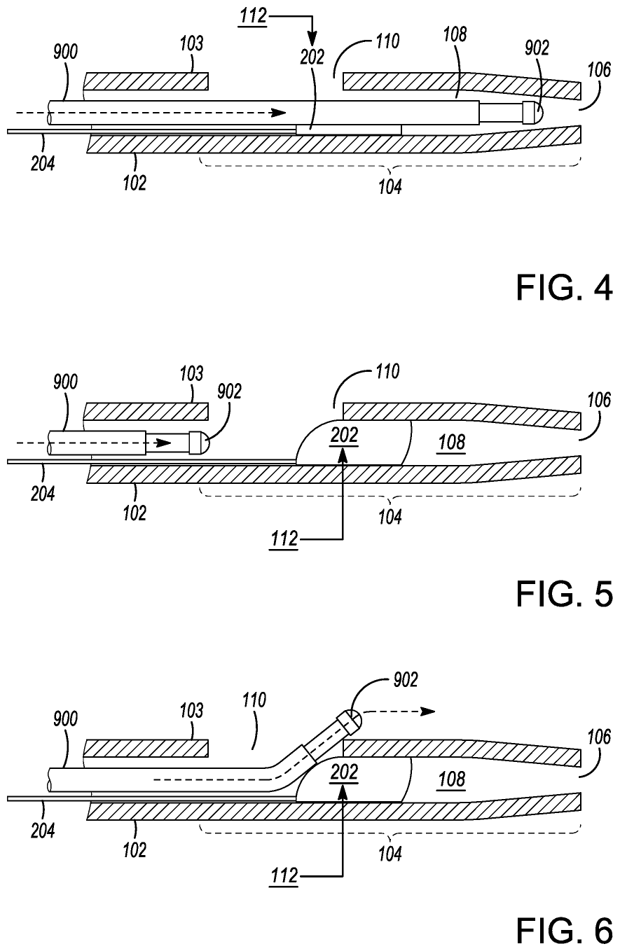

Referring to the embodiment (implementation) as depicted in FIG. 4, the guidewire deflector 112 is not activated (utilized). The distal guidewire section 902 (of the guidewire 900) reaches the axial portal 106, and may continue to be moved so that the distal guidewire section 902 may extend from the axial portal 106.

Referring to the embodiment (implementation) as depicted in FIG. 5, the guidewire deflector 112 is activated (utilized). Then, the distal guidewire section 902 (of the guidewire 900) is urged to move toward the axial portal 106, and the guidewire deflector 112 has been activated in the pathway of the distal guidewire section 902.

Referring to the embodiment (implementation) as depicted in FIG. 6, the distal guidewire section 902 (of the guidewire 900) reaches and contacts the guidewire deflector 112. The guidewire deflector 112, in use, deflects movement of the distal guidewire section 902 (of the guidewire 900) away from the axial portal 106 and toward the radial portal 110.

The distal guidewire section 902 may continue to be moved so that the distal guidewire section 902 may extend from the radial portal 110.

Referring to the embodiments (implementations) as depicted in FIG. 1 to FIG. 6, there is depicted a method for using the guidewire 900 having the distal guidewire section 902. The method includes using the guidewire deflector 112 to selectively deflect urged axial movement of the distal guidewire section 902 of the guidewire 900 away from an axial portal 106 and radially toward (and, preferably, through) the radial portal 110.

Referring to the embodiments (implementations) as depicted in FIG. 1 to FIG. 6, the distal guidewire section 902 (of the guidewire 900) is configured to selectively emit energy (such as radiofrequency energy, etc., and any equivalent thereof). For instance, the distal guidewire section 902 and the guidewire 900 may include a radiofrequency puncture device, such as the BAYLIS (TRADEMARK) POWERWIRE (REGISTERED TRADEMARK) radiofrequency guidewire manufactured by BAYLIS MEDICAL COMPANY (headquartered in Canada). In accordance with another embodiment, the distal guidewire section 902 includes (and is not limited to) a distal tip section presenting a mechanical cutting portion (for this case, the puncture is formed by physically moving the mechanical cutting portion into a biological feature).

Referring to the embodiments (implementations) as depicted in FIG. 1 to FIG. 6, the guidewire deflector 112 includes (preferably) the balloon assembly 202. The balloon assembly 202 may include any device configured to be inflatable and deflatable, expandable and collapsible (such as an expandable-and-contractible mesh structure or an expandable-and-collapsible mesh structure), etc. and any equivalent thereof. The balloon assembly 202 is, preferably, positioned and mounted to the inner wall (of the elongated catheter assembly 102) facing the catheter lumen 108, and positioned proximate to the axial portal 106 and the radial portal 110. The balloon assembly 202 is configured to direct (deflect) movement of the distal guidewire section 902 of the guidewire 900 toward, into and past the radial portal 110 (thereby preventing the distal guidewire section 902 from moving into and extending beyond the axial portal 106). The balloon assembly 202 may help to orient (assist in orientation of) the spatial orientation for the movement of the distal guidewire section 902 to a desired direction, etc. Advantageously, the guidewire deflector 112 may reduce (at least in part) a reliance on the characteristics of the guidewire 900 in order to assist the urged movement of the distal guidewire section 902 through the radial portal 110.

Referring to the embodiments (implementations) as depicted in FIG. 1 to FIG. 6, the balloon assembly 202 is (preferably) mounted to the distal section 104 of the elongated catheter assembly 102. The balloon assembly 202 is secured in the correct location (possibly by a gluing process or by function of geometry, etc.); that is, the balloon assembly 202 may be limited to travel so far due to a reduced inner diameter of the catheter lumen 108 (of the elongated catheter assembly 102). The shape of the balloon assembly 202 may allow for the guidewire 900 to ramp up against the balloon assembly 202 (or the guidewire deflector 112) and be directed to move out from the radial portal 110 (the side port). The balloon assembly 202 is configured to be inflated to a predetermined pressure; this may be adjustable or controlled, and to preferably avoid over-inflation to a bursting condition (causing risk to the patient, etc.). The balloon assembly 202 may be able to withstand the forces of compression from the elongated catheter assembly 102 and/or the force from the interaction with the guidewire 900 and/or the distal guidewire section 902.

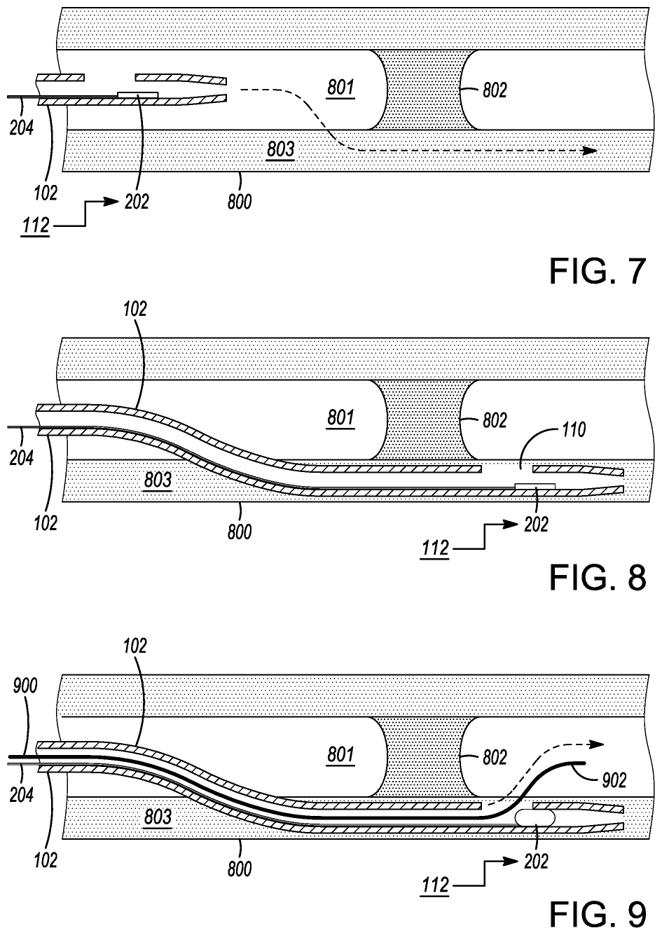

FIG. 7 to FIG. 9 depict cross-sectional views of embodiments (implementations) of the guidewire deflector 112 of FIG. 1. The cross-sectional views (FIG. 7 to FIG. 9) are taken along a cross-sectional line A-A of FIG. 1.

Referring to the embodiments (implementations) as depicted in FIG. 7 to FIG. 9, the elongated catheter assembly 102 is inserted into a biological tube 801 of a biological feature 800, and is then moved toward a biological blockage 802 (also called an occlusion) positioned in the biological tube 801. The biological blockage 802 prevents movement of the elongated catheter assembly 102. For this case, the elongated catheter assembly 102 is to moved into a side wall 803 (also called a subintimal space) of the biological feature 800, thereby bypassing the biological blockage 802, and then the elongated catheter assembly 102 may be urged to move around the biological blockage 802 and then back into the biological tube 801.

Referring to the embodiment (implementation) as depicted in FIG. 8, the elongated catheter assembly 102 is moved along the side wall 803 and just past the biological blockage 802. The elongated catheter assembly 102 is stopped from moving once the radial portal 110 is positioned just beyond (past) the biological blockage 802, and the radial portal 110 faces the biological tube 801.

Referring to the embodiment (implementation) as depicted in FIG. 9, the guidewire deflector 112 is activated. The guidewire 900 is moved toward the guidewire deflector 112. The guidewire deflector 112 deflects the distal guidewire section 902 (of the guidewire 900) toward the radial portal 110. The guidewire deflector 112 averts forward movement of the distal guidewire section 902 away from the axial portal 106 and toward the radial portal 110. The distal guidewire section 902 is further urged to move past the radial portal 110 and into the biological tube 801. Generally, for the case where the elongated catheter assembly 102 is utilized as a re-entry catheter, the distal guidewire section 902 is advanced beyond the biological blockage 802 (occlusion) along and inside the side wall 803 (also called a subintimal space). Once the distal guidewire section 902 is positioned proximate to the radial portal 110 (the side port), the guidewire deflector 112 (or the balloon assembly 202) is activated (inflated, etc.). The guidewire 900 is advanced so that the distal guidewire section 902 may be deflected (by the balloon assembly 202 once it is inflated) through (or pushed out from) the radial portal 110. If necessary, the distal guidewire section 902 may be utilized (such as, activated to emit energy) to puncture through the inner surface of the side wall 803 in order to gain access to the biological tube 801 (such as, a true lumen of a vessel, etc.). Advantageously, the guidewire deflector 112 may place less pressure on the vessel wall (the side wall 803), thereby imposing a physiologically less stressful approach. In accordance with FIG. 9, the balloon assembly 202 may help (to assist) in a re-entry movement of the distal guidewire section 902 from the side wall 803 back into the biological tube 801. The guidewire deflector 112 may help facilitate ease of directing the distal guidewire section 902 (of the guidewire 900) out of the elongated catheter assembly 102 (also called a re-entry type sheath, etc.). The guidewire deflector 112 may allow ease of advancement from the side wall 803 (subintimal channel) through the intimal layer and back into the biological tube 801 (the vasculature lumen). Advantageously, the distal guidewire section 902 (of the guidewire 900) may more easily move out of the radial portal 110 (of the elongated catheter assembly 102) by avoiding (at least in part) a need to manipulate the guidewire 900 (by rotating, pushing, pulling, etc.) in order to align the distal guidewire section 902 with the radial portal 110 (side port). Advantageously, the distal guidewire section 902, the guidewire deflector 112 may make it easier for the physician to orientate the guidewire 900 thereby allowing the distal guidewire section 902 to pass through the radial portal 110 (side port). Advantageously, the guidewire deflector 112 may allow the distal guidewire section 902 to be directed out from the radial portal 110 (side port) with fewer user maneuvering techniques. It will be appreciated that the guidewire deflector 112 may be utilized in various percutaneous coronary intervention therapies involving occlusions (such as chronic total occlusions). The combination of the elongated catheter assembly 102 with the guidewire deflector 112 (which may be called a re-entry device) may also be used to navigate to side branches of vessels, etc. For instance, accessing the coronaries with the guidewire 900 may be challenging; therefore, using the combination of the elongated catheter assembly 102 with the guidewire deflector 112 may offer a more reliable way to gain access to the coronaries of the patient.

Referring to the embodiment (implementation) as depicted in FIG. 9, the elongated catheter assembly 102 may be configured as a steerable catheter, if so desired.

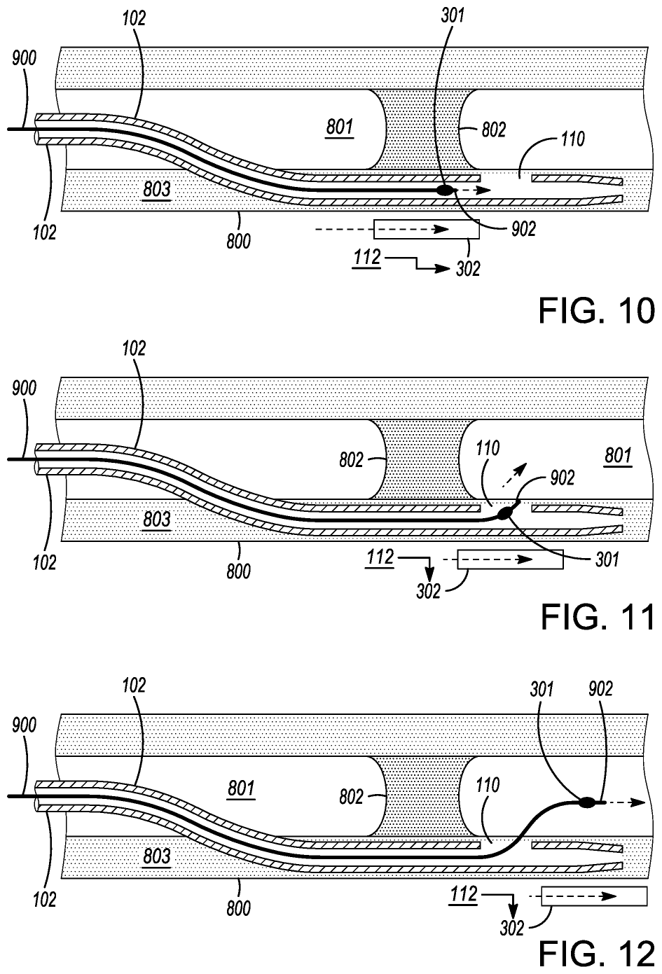

FIG. 10 to FIG. 12 depict cross-sectional views of embodiments (implementations) of the guidewire deflector 112 of FIG. 1. The cross-sectional views (FIG.10 to FIG. 12) are taken along a cross-sectional line A-A of FIG. 1.

Referring to the embodiments (implementations) as depicted in FIG. 10 to FIG. 12, the guidewire deflector 112 includes a first magnet 301 and a second magnet 302. The first magnet 301 is mounted to the distal guidewire section 902 (of the guidewire 900). The second magnet 302 is movable relative to the first magnet 301. The second magnet 302 is movable along the side wall 803 (of the biological feature 800) from one side of the biological blockage 802 to the other side of the biological blockage 802. The first magnet 301 includes a permanent magnet. The second magnet 302 includes an electromagnetic device configured to selectively polarize the second magnet 302 to be (A) attractive to the first magnet 301, or (B) repulsive to the first magnet 301. It will be appreciated that a medical-imaging system (known and not depicted) may be utilized for generating images of components of the elongated catheter assembly 102 and the biological feature 800; in this way, the images of the biological feature 800 and the elongated catheter assembly 102 may be displayed to the user during the procedure.

Referring to the embodiment (implementation) as depicted in FIG. 10, the second magnet 302 and the first magnet 301 are configured to be magnetically attracted to each other (one another). This is done in such a way that the second magnet 302 magnetically pulls the first magnet 301 past the biological blockage 802 (through the side wall 803).

Referring to the embodiment (implementation) as depicted in FIG. 11, the second magnet 302 and the first magnet 301 are configured to be magnetically repelled (repulsive) from each other. Once the distal guidewire section 902 is positioned proximate to the radial portal 110, the second magnet 302 magnetically pushes (repulses) the first magnet 301 to move toward, and through, the radial portal 110 and into the biological tube 801. It will be appreciated that the first magnet 301 includes a permanent magnet. The second magnet 302 includes an electromagnetic device configured to selectively polarize the second magnet 302 to be (A) attractive to the first magnet 301 in such a way that the second magnet 302 magnetically pulls the first magnet 301 toward the second magnet 302, or (B) repulsive to the first magnet 301 in such a way that the second magnet 302 magnetically pushes the first magnet 301 away from the second magnet 302. It will be appreciated that, generally, the first magnet 301 and the second magnet 302 are configured to be: (A) selectively attractive to each other; this is done, preferably, in such a way that the second magnet 302 magnetically pulls the first magnet 301 toward the second magnet 302, and/or (B) selectively repulsive to each other (one another); this is done, preferably, in such a way that the second magnet 302 magnetically pushes the first magnet 301 away from the second magnet 302.

Referring to the embodiment (implementation) as depicted in FIG. 12, the second magnet 302 and the first magnet 301 are configured to be magnetically attracted to each other (one another). This is done, preferably, in such a way that the second magnet 302 magnetically pulls the first magnet 301 along the biological tube 801.

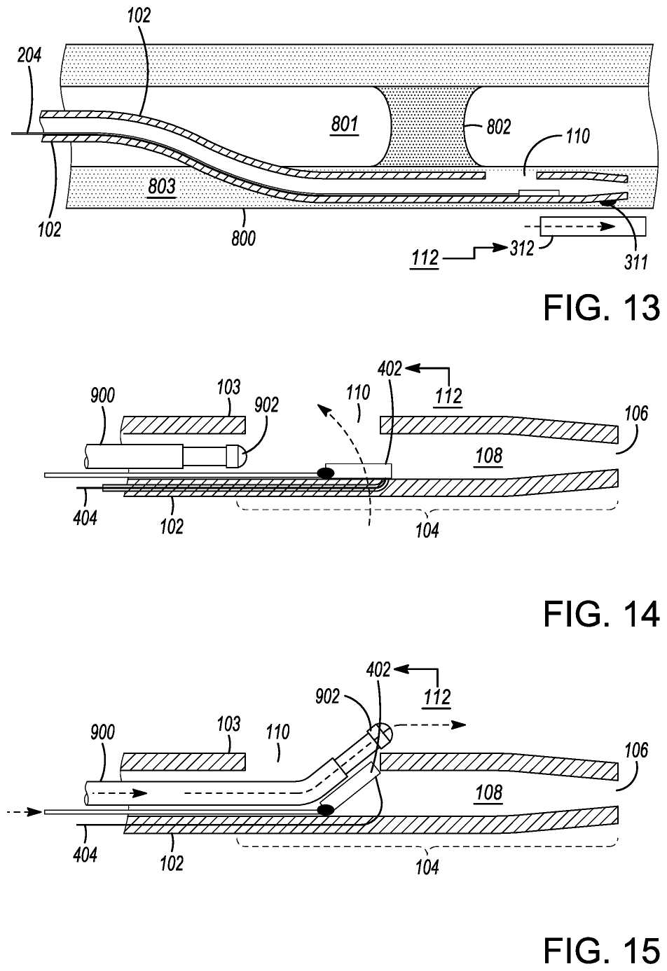

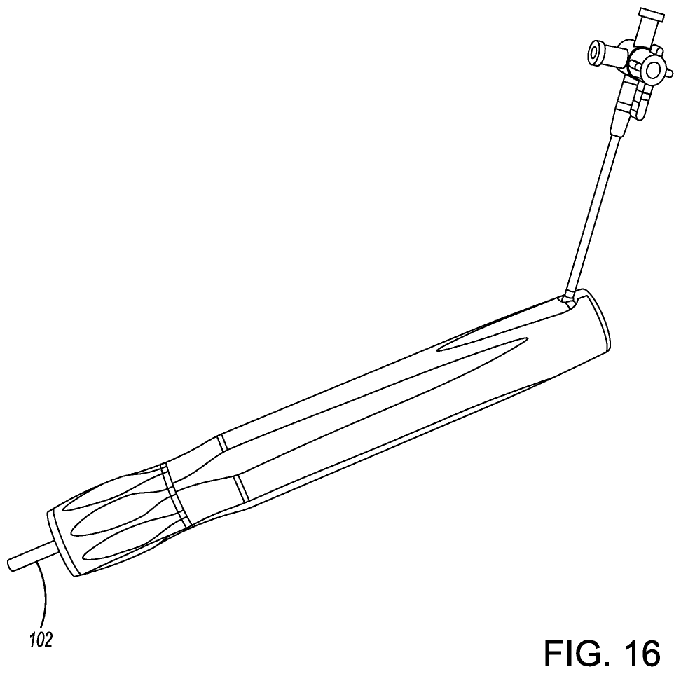

FIG. 13 to FIG. 16 depict cross-sectional views (FIG. 13 to FIG. 15) of embodiments (implementations) of the guidewire deflector 112 of FIG. 1, and a side view (FIG. 16) of embodiments (implementations) of the elongated catheter assembly 102 of FIG. 1. The cross-sectional views (FIG. 13 to FIG. 15) are taken along a cross-sectional line A-A of FIG. 1.

Referring to the embodiment (implementation) as depicted in FIG. 13, the guidewire deflector 112 includes a first magnet device 311 and a second magnet device 312. The first magnet device 311 is mounted to the distal section 104 (of the elongated catheter assembly 102). The second magnet device 312 is movable relative to the first magnet device 311. The second magnet device 312 is movable along the side wall 803 (of the biological feature 800) from one side of the biological blockage 802 to the other side of the biological blockage 802. The first magnet device 311 includes a permanent magnet. The second magnet device 312 includes an electromagnetic device configured to selectively polarize the second magnet device 312 to be (A) attractive to the first magnet device 311, or (B) repulsive to the first magnet device 311. It will be appreciated that a medical-imaging system (known and not depicted) may be utilized for generating images of components of the elongated catheter assembly 102 and the biological feature 800; in this way, the images of the biological feature 800 and the elongated catheter assembly 102 may be displayed to the user during the procedure. The second magnet device 312 is configured to magnetically attract the first magnet device 311 along the side wall 803. Generally, the first magnet device 311 and the second magnet device 312 are configured to: (A) be selectively attractive to each other; or (B) be selectively repulsive to each other (one another).

Referring to the embodiments (implementations) as depicted in FIG. 14 and FIG. 15, the guidewire deflector 112 includes a plate element 402 and a control wire 404. The plate element 402 is pivotally mounted to the inner surface facing the catheter lumen 108. The plate element 402 is positioned (mounted) proximate to the axial portal 106 and the radial portal 110. The control wire 404 is attached to the plate element 402. The control wire 404 is configured to selectively move the plate element 402 between a plate-activated position (as depicted in FIG. 15) and a plate-storage position (as depicted in FIG. 14). The plate element 402 is configured to be pivotally moved between the plate-activated position (as depicted in FIG. 15) and the plate-storage position (as depicted in FIG. 14) in response to urged movement of the control wire 404. The plate element 402 is configured to selectively deflect the urged movement of the distal guidewire section 902 (of the guidewire 900) toward the radial portal 110 (and away from the axial portal 106) after (once) the plate element 402 is activated; that is, once the plate element 402 is selectively moved from the plate-storage position (as depicted in FIG. 14) to the plate-activated position (as depicted in FIG. 15) in response to urged movement of the control wire 404.

Referring to the embodiment (implementation) as depicted in FIG. 16, the distal section 104 of the elongated catheter assembly 102 is configured to be spatially adjustable and/or orientable. In this arrangement, the axial portal 106 and the radial portal 110 may be oriented to face a desired direction.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus for use with a guidewire having a distal guidewire section, comprising:
an elongated catheter assembly defining a catheter lumen being configured to receive the distal guidewire section of the guidewire, and also defining an axial portal and a radial portal; and
a guidewire deflector being mounted to the elongated catheter assembly, and the guidewire deflector being configured to selectively deflect urged axial movement of the distal guidewire section of the guidewire away from the axial portal and radially toward the radial portal.

2. An apparatus for use with a guidewire having a distal guidewire section, comprising:
an elongated catheter assembly defining a catheter lumen extending axially along the elongated catheter assembly; and
the catheter lumen being configured to receive the distal guidewire section of the guidewire; and
the elongated catheter assembly defining an axial portal extending axially from the catheter lumen; and
the elongated catheter assembly also defining a radial portal extending radially from the catheter lumen; and
a guidewire deflector mounted in the catheter lumen; and
the guidewire deflector being configured to selectively deflect urged axial movement of the distal guidewire section of the guidewire being made to travel axially along the catheter lumen away from the axial portal and radially toward the radial portal.

3. The apparatus of claim 2, wherein:
the elongated catheter assembly has a sidewall; and
the elongated catheter assembly also has a distal section extending from the sidewall.

4. The apparatus of claim 2, wherein:
the catheter lumen extends axially toward and through a distal section of the elongated catheter assembly to the axial portal.

5. The apparatus of claim 2, wherein:
a distal section of the elongated catheter assembly is configured to permit movement of the distal guidewire section of the guidewire travelling axially along the catheter lumen toward, and through, the axial portal; and
the distal section defines the radial portal extending radially from the catheter lumen; and
the radial portal extends radially through a sidewall of the elongated catheter assembly.

6. The apparatus of claim 2, wherein:
the guidewire deflector is mounted in the catheter lumen at a distal section of the elongated catheter assembly; and
the guidewire deflector is mounted proximate to the axial portal.

7. The apparatus of claim 2, wherein:
a proximal section of the catheter lumen is configured to receive the distal guidewire section of the guidewire; and
a distal section of the catheter lumen defines the axial portal and the radial portal.

8. The apparatus of claim 2, wherein:
the catheter lumen, the axial portal and the radial portal are in fluid communication with each other.

9. The apparatus of claim 2, wherein:
the guidewire deflector is user controllable.

10. The apparatus of claim 2, wherein:
the guidewire deflector includes:
a balloon assembly configured to be selectively inflated and deflated; and
an inflation lumen extending along a length of the elongated catheter assembly; and
the inflation lumen being in fluid communication with the balloon assembly; and
the inflation lumen being configured to be fluidly connected to an inflation source.

11. The apparatus of claim 2, wherein:
the guidewire deflector includes:
a first magnet mounted to the distal guidewire section of the guidewire; and
a second magnet being movable relative to the first magnet.

12. The apparatus of claim 11, wherein:
the first magnet includes a permanent magnet; and
the second magnet includes an electromagnetic device being configured to selectively polarize the second magnet to be:
attractive to the first magnet in such a way that the second magnet magnetically pulls the first magnet toward the second magnet; and
repulsive to the first magnet in such a way that the second magnet magnetically pushes the first magnet away from the second magnet.

13. The apparatus of claim 11, wherein:
the first magnet and the second magnet are configured to be:
selectively attractive to each other in such a way that the second magnet magnetically pulls the first magnet toward the second magnet; and
selectively repulsive to each other in such a way that the second magnet magnetically pushes the first magnet away from the second magnet.

14. The apparatus of claim 2, wherein:
the guidewire deflector includes:
a first magnet device mounted to a distal section of the elongated catheter assembly; and
a second magnet device being movable relative to the first magnet device.

15. The apparatus of claim 14, wherein:
the first magnet device includes a permanent magnet; and
the second magnet device includes an electromagnetic device being configured to selectively polarize the second magnet device to be:
attractive to the first magnet device; and
repulsive to the first magnet device.

16. The apparatus of claim 14, wherein:
the first magnet device and the second magnet device are configured to be:
selectively attractive to each other; and
selectively repulsive to each other.

17. The apparatus of claim 2, wherein:
the guidewire deflector includes:
a plate element being pivotally mounted to an inner surface facing the catheter lumen; and
the plate element being positioned proximate to the axial portal and the radial portal; and
a control wire being attached to the plate element; and
the control wire being configured to selectively move the plate element between a plate-activated position and a plate-storage position; and
the plate element being configured to be pivotally moved between the plate-activated position and the plate-storage position in response to urged movement of the control wire.

18. The apparatus of claim 17, wherein:
the plate element is configured to selectively deflect urged movement of the distal guidewire section of the guidewire toward the radial portal and away from the axial portal after the plate element is selectively moved from the plate-storage position to the plate-activated position in response to urged movement of the control wire.

19. A method of using a guidewire having a distal guidewire section, the method comprising:
actuating a guidewire deflector being mounted to an elongated catheter assembly defining a catheter lumen being configured to receive the distal guidewire section of the guidewire, and also defining an axial portal and a radial portal, to selectively deflect urged axial movement of the distal guidewire section of the guidewire away from the axial portal and radially toward the radial portal.

20. The method of claim 19, wherein the guidewire deflector includes one or more balloon, magnet, and plate.
